(19) United States
(12) Patent Application Publication
(10) Pub. No.: US 2026/0335460 A1
(43) Pub. Date: Oct. 29, 2026

(54) METHODS AND COMPOSITIONS FOR IMPROVING RESPONSE TO IMMUNOTHERAPY

(71) Applicant: The Brigham and Women's Hospital, Inc., Boston, MA (US)

(72) Inventors: Tanya MAYADAS-NORTON, Jamaica Plain, MA (US); Huan WANG, Newton, MA (US)

(21) Appl. No.: 19/119,672

(22) PCT Filed: Oct. 13, 2023
(86) PCT No.: PCT/US2023/076913

§ 371 Date: Apr. 9, 2025

Related U.S. Application Data
(60) Provisional application No. 63/415,926, filed on Oct. 13, 2022.

(51) Int. Cl. A61K 47/68; A61P 35/00; C07K 14/47; C07K 16/28

(52) U.S. Cl. CPC A61K 47/6849; A61P 35/00; C07K 14/4748; C07K 16/2818; C07K 16/283

(57) ABSTRACT

Described herein are methods and compositions for use in generating or promoting an immune response to cancer, comprising administering a combination of an immunotherapy using an immune checkpoint inhibitor, e.g., anti-PD-1 antibodies, and a conjugate comprising an antigen and an anti-FcγRIIIB antibody.

### CLAIM OF PRIORITY

This application claims the benefit of U.S. Provisional Patent Application Ser. No. 63/415,926, filed on Oct. 13, 2022. The entire contents of the foregoing are hereby incorporated by reference.

### TECHNICAL FIELD

Described herein are methods and compositions for use in generating or promoting an immune response to cancer, comprising administering a combination of an immunotherapy using an immune checkpoint inhibitor, e.g., anti-PD-1 antibodies and a conjugate comprising an antigen and an anti-FcγRIIIB antibody.

### BACKGROUND

While immunotherapy using immune checkpoint inhibitors (ICIs) is a promising treatment for many cancers, its success has been limited in “cold” tumors that lack T-cell infiltration (Liu and Sun, Theranostics. 2021; 11(11): 5365-5386).

### SUMMARY

The present invention is based, at least in part, on the discovery that Anti-CD16B-antigen conjugates (AACs) binding neutrophils can drive T cells into tumor tissues, turning “cold” tumors into “hot” tumors that respond to treatment with anti-PD1.

Thus, provided herein are compositions comprising (i) a conjugate comprising a tumor antigen and an antibody comprising an antigen-binding domain that binds to FcγRIII, optionally wherein the conjugate is a fusion protein or chemical conjugate, and (ii) an immune checkpoint inhibitor (ICI). In some embodiments, the tumor antigen is listed in Table A.

In some embodiments, the one or more ICIs are selected from an antibody that binds to PD-1, CD40, PD-L1, Tim3, Lag3, CTLA-4, or T-cell immunoglobulin and ITIM domains (TIGIT). In some embodiments, the ICI is an antibody that binds PD-1.

Also provided are the compositions described herein for use in a method of treating a subject who has cancer. In some embodiments, the subject has a solid tumor. In some embodiments, the solid tumor does not have infiltrating leukocytes, or is immunologically cold. In some embodiments, the solid tumor is melanoma, pancreatic cancer, prostrate cancer, colon cancer, glioblastoma, or ovarian cancer.

Also provided herein are methods of treating a subject who has cancer, the method comprising administering to the subject an effective amount of a composition described herein.

Further, provided herein are methods of treating a subject who has cancer, comprising administering to the subject an effective amount of (i) a conjugate comprising an antigen and an antibody comprising an antigen-binding domain that binds to FcγRIII, optionally wherein the conjugate is a fusion protein or chemical conjugate, and (ii) an immune checkpoint inhibitor (ICI). In some embodiments, the tumor antigen is shown in Table A. In some embodiments, the one or more ICIs are selected from an antibody that binds to PD-1, CD40, PD-L1, Tim3, Lag3, CTLA-4, or T-cell immunoglobulin and ITIM domains (TIGIT). In some embodiments, the ICI is an antibody that binds PD-1.

In some embodiments, the subject has a solid tumor. In some embodiments, the tumor does not have infiltrating leukocytes, or is immunologically cold. In some embodiments, the tumor is melanoma, pancreatic cancer, prostrate cancer, glioblastoma, colon cancer, or ovarian cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGs., and from the claims.

### DESCRIPTION OF DRAWINGS

FIGS. 1A-B. AAC but not anti-PD-1 increases early T cell and Natural Killer (NK) cell accumulation in an established B16F10-Ova melanoma. a. Schematic for the timeline of indicated treatments, hFcγR, humanized FcγR mice expressing FcγRIIIB (CD16B) and FcγRIIA (CD32A) selectively on neutrophils of mice lacking their endogenous activating FcγRs (Tsuboi et al. Immunity 2008, 28:833). B16F10-Ova, murine melanoma cells expressing Ovalbumin (Ova), a model T cell-dependent antigen. OT-I, naïve Ova-specific CD8⁺ T cells. AAC, anti-FcγRIIIB conjugated to Ova. IgG-Ova, non-targeting isotype of anti-FcγRIIIB conjugated to Ova. αPD1, anti-PD1. Iso^{P}, isotype control of anti-PD-1. b. Tumor-infiltrating CD45⁺ leukocytes, CD8⁺, CD4⁺ T cells and NK cells in indicated treatment groups of mice at day 12 after tumor inoculation identified by flow cytometry. N=9-12 per group. Data are means±SEM. *p<0.05, **p<0.01, ***p<0.001, ****p<0.0001, one-way ANOVA with Tukey multiple comparison test.

FIGS. 2A-B. AAC reduces tumor growth independently and significantly enhances the efficacy of anti-PD-1. a. Schematic for the timeline of indicated treatments, hFcγR, humanized FcγR mice expressing FcγRIIIB (CD16B) and FcγRIIA (CD32A) selectively on neutrophils of mice lacking their endogenous activating FcγRs (Tsuboi et al. Immunity 2008, 28:833). B16F10-Ova, murine melanoma cells expressing Ovalbumin (Ova), a model T cell-dependent antigen. OT-I, naïve Ova-specific CD8⁺ T cells. AAC, anti-FcγRIIIB conjugated to Ova. IgG-Ova, non-targeting isotype of anti-FcγRIIIB conjugated to Ova. «PD1, anti-PD1. Iso^{P}, isotype control of anti-PD-1. b. Bar graph (left panel) of tumor volume at day 19 normalized to the average in IgG-Ova+Iso^{P} control group within each experiment. The number of mice per group (n=11-38 per group) are in parentheses. Spider plots (right panel) showing growth of tumors in individual mice over time in indicated treatment groups of mice. Data are means±SEM. *p<0.05, **p<0.01, ***p<0.001, ****p<0.0001, One-way ANOVA with Tukey multiple comparison test.

FIGS. 3A-C. Anti-PD-1 in combination with AAC increases tumor accumulation of antigen-specific effector T cells, and T_{SCM} and T_{RM} memory T cells. Tumors were harvested on day 19 and analyzed by flow cytometry for surface and intracellular T cell markers and the number of cells per mm³ volume of tumor is given. a. Ova-specific CD8⁺ T cells detected with MHC-I tetramers (Tet*). b. Tumor-infiltrating Ova-specific CD8⁺ T cells positive for granzyme B, perforin, IFNγ and TNFα. c. T_{SCM} and T_{RM} of Ova-specific CD8⁺ T cells. N=6-13 per group. Data are means±SEM. *p<0.05, **p<0.01, ***p<0.001, ****p<0.0001, One-way ANOVA with Tukey multiple comparison test.

### DETAILED DESCRIPTION

Immune checkpoint inhibitors are largely ineffective in patients with “cold” tumors as their efficacy relies on unleashing preexisting CD8 T cells within the tumor.

The treatment of tumors with an anti-FcgRIII (CD16B)-antigen conjugate (AAC) that engages neutrophils (WO 2020/154424 and Mysore et al., Nat Commun 2021) has been shown to convert neutrophils into robust antigen presenting cells and induce anti-tumor immunity upon prophylactic treatment of a B16F10 melanoma in mice. B16F10 melanoma in mice has been shown to be a “cold” tumor with minimal infiltration with T cells, NK cells and other leukocytes with anti-tumor functions due to an immunosuppressive microenvironment (Urs et al., 2019, available online at biopharma.labcorp.com/industry-solutions/by-therapeutic-area/oncology/preclinical/tumor-spotlights/b16-f10-a-murine-melanoma-model.html). As shown herein, therapeutic AAC treatment of an established B16F10-Ova melanoma, resulted in a marked increase in cytotoxic T cells and NK cells with activation markers and other immune cells, thereby turning an immunologically “cold” tumor “hot”. The present inventors hypothesized that the efficacy of checkpoint inhibitors would be improved when combined with AAC treatment.

As shown herein, AAC in combination with a checkpoint inhibitor (in these examples, anti-PD-I was used) lead to a significant reduction in tumor growth while anti-PD-1 alone resulted in only partial, non-significant reduction in tumor growth. The combination therapy of AAC plus anti-PD-1 lead to a larger increase in leukocyte infiltration compared to anti-PD-1 or AAC alone. AAC therapeutic treatment of an established B16F10-Ova melanoma reduced tumor growth and significantly increased the intratumoral infiltration of immune cells including activated CD8⁺ and CD4⁺ T cells, NK cells and T_{SCM} and T_{RM} memory T cells, which are known to associate with anti-tumor immunity. In addition, AAC administration in combination with anti-PD1 increased epitope spreading; in a mouse model of “cold” melanomas, spreading of epitope recognition toward wild-type melanocyte antigens has been shown to be associated with markedly improved anti-PD-1 efficacy (Lo et al., Sci Transl Med. 2021 Feb. 17; 13(581):eabd8636).

Thus, AAC enhances the efficacy of an immune checkpoint inhibitor, anti-PD-1. AAC targeting neutrophils combined with an immune checkpoint inhibitor such as anti-PD-1 represent a new immunotherapy for the treatment of solid tumors. The combination should improve the efficacy of anti-PD1 and other immune checkpoint inhibitor in all tumors for which they are FDA approved (Vaddepally et al Cancers 2020, 12(3):738) as well as immunologically “cold” solid tumors resistant to immune checkpoint inhibitors. This especially includes pancreatic (Chick et al Cancers 2023, 15(15):3967), prostrate (Sridaran et al Cell Rep Med 2023 September 15:101199), colon (Puccini et al J Immunother Cancer 2020, e000404), glioblastoma (Dapash et al Cancers 2021, 13(18):4548) and ovarian (Le Saux et al. Semin Cancer Biol 2021, 77:127-143) cancer.

Thus, described herein are methods for treating subjects with cancer. As used in this context, to “treat” cancer means to ameliorate at least one symptom of the cancer. Administration of a therapeutically effective amount of a compound described herein for the treatment of a cancer can result in decreased or stabilized tumor burden, decreased or stabilized tumor size, decreased or stabilized tumor growth rate, decreased or stabilized tumor serum markers, and decreased or stabilized risk of metastasis. The subjects can be, e.g., mammals, e.g., human or veterinary subjects. Although humans are used as examples herein, other mammals can also be treated using the present methods, with species-appropriate antibodies and other reagents.

The methods generally include identifying a subject who has a tumor, e.g., a cancer. As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer will be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. In general, the methods described herein can be practiced on subjects with solid tumors or hematopoietic tumors, which are malignancies of cells of the immune system. Methods for identifying or diagnosing subjects with cancers are known in the art.

Tumors include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, cancers evaluated or treated by the methods described herein include epithelial cancers, such as a lung cancer (e.g., non-small-cell lung cancer (NSCLC)), breast cancer, colorectal cancer, kidney cancer, head and neck cancer, prostate cancer, or ovarian cancer. Epithelial malignancies are cancers that affect epithelial tissues.

In some embodiments, the cancers treated by the combination methods described herein are solid tumors for which immunotherapy using immune checkpoint inhibitors (e.g., anti-PD1 and others) are FDA approved, as these methods can be used to improve efficacy of these modalities. In some embodiments, the combination methods described could make immunologically “cold” solid tumors responsive to the above modalities. This includes pancreatic (Chick et al Cancers 2023, 15(15):3967), prostrate (Sridaran et al Cell Rep Med 2023 September 15:101199), colon (Puccini et al J Immunother Cancer 2020, e000404), glioblastoma (Dapash et al Cancers 2021, 13(18):4548) and ovarian (Le Saux et al. Semin Cancer Biol 2021, 77:127-143) cancer. Methods for identifying cold tumors include histological analysis of patient biopsies immunostained with antibodies to examine the degree and type of immune cell infiltration (e.g., CD8+ T cells and NK cells) (Wang et al., MedComm 2020, 4(5): e343).

### Anti-FcγRIIIB (CD16B) Antibodies Conjugated to Antigen

Provided herein are methods in which a construct comprising an anti-FcγRIIIB (CD16B) antibody conjugated to a tumor antigen is used, either in vitro/ex vivo to prepare cells for use in a cell therapy method, or in vivo to stimulate an immune response to the antigen. In some embodiments, the conjugate is delivered intravenously or intratumorally to a subject, in addition to treatment with anti-PD1 antibodies.

In the present methods, a construct comprising an anti-FcγRIIIB antibody conjugated to a tumor antigen is used. The term “antigen” comprises any structure that is capable of inducing an immune response in an organism either by itself or when coupled to a suitable carrier molecule or cell. Therefore, antigens according to the present invention include low molecular compounds which serve as haptens as well as whole cells such as tumor cells as well as the parts thereof such as polypeptides, oligopeptides derived therefrom, lipids such as glycolipids, polysaccharides and nucleic acids. Tumor antigens can include, e.g., a Cancer Testis (CT) antigen, a protein that is normally expressed only on human germ line cells but is also present in a subset of malignant tumors, e.g., a CT antigen listed in Table A, or a neoantigen that arises from tumor-specific mutations. Neoantigens can arise from any genomic mutation altering protein sequence. See, e.g., Hu, Ott, Wu “Towards personalized, tumor-specific, therapeutic vaccines for cancer.” Nat Rev Immunol. 2018 March; 18(3):168-182; Vigneron et al., Cancer Immun. 2013; 13:15; Hutchison and Pritchard, Mamm Genome. 2018; 29(11): 714-730; Teku and Vihinen, Sci Rep. 2018; 8:12735; Renkvist et al., Cancer Immunology and Immunotherapy 2001; 50:3-15.

TABLE A
Tumor antigens (CT Antigens)

| | | |
|---|---|---|
| MAGEA1 | SPANXC | LDHC |
| MAGEA2 | SPANXD | MORC1 |
| MAGEA3 | SPANXN1 | DKKL1 |
| MAGEA4 | SPANXN2 | SPO11 |
| MAGEA5 | SPANXN3 | CRISP2 |
| MAGEA6 | SPANXN4 | FMR1NB |
| MAGEA8 | SPANXN5 | FTHL17 |
| MAGEA9 | XAGE1D | NXF2 |
| MAGEA10 | XAGE1C | TAF7L |
| MAGEA11 | XAGE1B | TDRD1 |
| MAGEA12 | XAGE1 | TDRD6 |
| BAGE | XAGE2 | TDRD4 |
| BAGE2 | XAGE3 | TEX15 |
| BAGE3 | XAGE-3b | FATE1 |
| BAGE4 | XAGE-4/RP11-167P23.2 | TPTE |
| BAGE5 | XAGE5 | CT45A1 |
| MAGEB1 | DDX43 | CT45A2 |
| MAGEB2 | SAGE1 | CT45A3 |
| MAGEB5 | ADAM2 | CT45A4 |
| MAGEB6 | PAGE5 | CT45A5 |
| MAGEB3 | CT16.2 | CT45A6 |
| MAGEB4 | PAGE1 | HORMAD1 |
| GAGE1 | PAGE2 | HORMAD2 |
| GAGE2A | PAGE2B | CT47A1 |
| GAGE3 | PAGE3 | CT47A2 |
| GAGE4 | PAGE4 | CT47A3 |
| GAGE5 | LIPI | CT47A4 |
| GAGE6 | VENTXP1 | CT47A5 |
| GAGE7 | IL13RA2 | CT47A6 |
| GAGE8 | TSP50 | CT47A7 |
| SSX1 | CTAGE1 | CT47A8 |
| SSX2 | CTAGE-2 | CT47A9 |
| SSX2b | CTAGE5 | CT47A10 |
| SSX3 | SPA17 | CT47A11 |
| SSX4 | ACRBP | CT47B1 |
| CTAG1B | CSAG1 | SLCO6A1 |
| LAGE-1b | CSAG2 | TAG |
| CTAG2 | DSCR8 | LEMD1 |
| MAGEC1 | MMA1b | HSPB9 |
| MAGEC3 | DDX53 | CCDC110 |
| SYCP1 | CTCFL | ZNF165 |
| BRDT | LUZP4 | SPACA3 |
| MAGEC2 | CASC5 | CXorf48 |
| SPANXA1 | TFDP3 | THEG |
| SPANXB1 | JARID1B | ACTL8 |
| NY-ESO-1 | SEMG1 | FBXO39 |
| NLRP4 | POTED | RGS22 |
| COX6B2 | POTEE | cyclin Al |
| LOC348120 | POTEA | C15orf60 |
| CCDC33 | POTEG | CCDC83 |
| LOC196993 | POTEB | TEKT5 |
| PASD1 | POTEC | NR6A1 |
| LOC647107 | POTEH | TMPRSS12 |
| TULP2 | GOLGAGL2 FA | TPPP2 |
| CT66/AA884595 | CDCA1 | PRSS55 |
| PRSS54 | PEPP2 | DMRT1 |
| RBM46 | OTOA | EDAG, NDR |
| CT69/BC040308 | CCDC62 | DNAJB8 |
| CT70/BI818097 | GPATCH2 | CSAG3B |
| SPINLW1 | CEP55 | CTAG1A |
| TSSK6 | FAM46D | GAGE12B |
| ADAM29 | TEX14 | GAGE12C |
| CCDC36 | CTNNA2 | GAGE12D |
| LOC440934 | FAM133A | GAGE12E |
| SYCE1 | LOC130576 | GAGE12F |
| CPXCR1 | ANKRD45 | GAGE12G |
| TSPY3 | ELOVL4 | GAGE12H |
| TSGA10 | IGSF11 | GAGE12I |
| HIWI, MIWI, PIWI | TMEFF1 | GAGE12J |
| PIWIL2 | TMEFF2 | GAGE13 |
| ARMC3 | ARX | LOC728137 |
| AKAP3 | SPEF2 | MAGEA2B |
| Cxorf61 | GPAT2 | MAGEA9B/LOC728269 |
| PBK | TMEM108 | NXF2B |
| C21orf99 | NOL4 | SPANXA2 |
| OIP5 | PTPN20A | SPANXB2 |
| CEP290 | SPAG4 | SPANXE |
| CABYR | MAEL | SSX4B |
| SPAG9 | RQCD1 | SSX5 |
| MPHOSPH1 | PRAME | SSX6 |
| ROPN1 | TEX101 | SSX7 |
| PLAC1 | SPATA19 | SSX9 |
| CALR3 | ODF1 | TSPY1D |
| PRM1 | ODF2 | TSPY1E |
| PRM2 | ODF3 | TSPY1F |
| CAGE1 | ODF4 | TSPY1G |
| TTK | ATAD2 | TSPY1H |
| LY6K | ZNF645 | TSPY1I |
| IMP-3 | MCAK | TSPY2 |
| AKAP4 | SPAG1 | XAGE1E |
| DPPA2 | SPAG6 | XAGE2B/CTD- |
| KIAA0100 | SPAG8 | 2267G17.3 |
| DCAF12 | SPAG17 | |

Where neoantigens are used, preferably the neoantigens expressed in an individual subject are identified and used, e.g., as described in Hu et al., Nat Rev Immunol. 2018 March; 18(3):168-182; Ott et al., Nature. 2017 Jul. 13; 547(7662):217-221; Carreno et al., Science. 2015 348(6236):803-808; and Linette and Carreno, Trends Mol Med. 2017 October; 23(10):869-871.

Anti-FcγRIIIB antibodies are known in the art, as are methods of making them. Anti-FcγRIIIB antibodies are commercially available, e.g., from Abbexa Ltd; Abcam; Abeomics; antibodies-online; Aviva Systems Biology; Biogems International, Inc.; BioLegend; Biorbyt; CEDARLANE; Cell Sciences; Creative Diagnostics; Elabscience Biotechnology Inc.; EXBIO Praha, a.s.; GeneTex; Invitrogen Antibodies; LifeSpan BioSciences; MBL International; Miltenyi Biotec; MyBioSource.com; NSJ Bioreagents; OriGene Technologies; Peninsula Laboratories International, Inc.; ProSci, Inc; R&D Systems; Santa Cruz Biotechnology, Inc.; Signalway Antibody LLC; Sino Biological, Inc.; SouthernBiotech; STEMCELL Technologies, Inc.; and United States Biological. In some embodiments, the antibody is 3G8 (e.g., available from biolegend, Santa Cruz Biotechnology, and others; see, e.g., Perussia and Trinchieri, J Immunol. 1984 March; 132(3):1410-5). These antibodies can be used as-is or modified, e.g., to reduce immunogenicity or alter half-life.

In some embodiments, the antibody also recognizes FcγRIIIA (CD16a: a transmembrane isoform of the GPI-linked FcγRIIIB, CD16b) that is present on macrophages and NK cells. In some embodiments, the antibody is a FcγRIIIB specific antibody that does not bind to FcγRIIIA.

### Antibodies

The term “antibody” as used herein refers to an immunoglobulin molecules, preferably IgG, as well as modified forms, or antigen-binding fragments, variants, or derivatives thereof. The antibody can be polyclonal, monoclonal, recombinant, chimeric, de-immunized or humanized, fully human, non-human, (e.g., murine), or single chain antibody. In some embodiments the antibody has effector function and can fix complement. Methods for making antibodies and fragments thereof are known in the art, see, e.g., Harlow et. Al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1^{st} edition, Dec. 13, 2006); Kontermann and Dübel, Antibody Engineering Volume 1 (Springer Protocols) (Springer; 2^{nd} ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010). When antibodies are obtained by the phage display technique, surface plasmon resonance as employed in the BIAcore system can be used to increase the efficiency of phage antibodies which bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the production of humanized antibodies are described in, e.g., European application EP-A1 0 239 400 and international application WO90/07861. A further source of antibodies to be utilized in accordance with the present invention are so-called xenogeneic antibodies. The general principle for the production of xenogeneic antibodies such as human-like antibodies in mice is described in, e.g., international applications WO91/10741, WO94/02602, WO96/34096 and WO 96/33735.

Modified forms of antibodies, or antigen-binding fragments, variants, or derivatives thereof described herein can be made from whole precursor or parent antibodies using techniques known in the art. Exemplary techniques are discussed in more detail herein. Antibodies, or antigen-binding fragments, variants, or derivatives thereof described herein can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Antibodies, or antigen-binding fragments, variants, or derivatives thereof described herein also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In particular embodiments, antibodies, or antigen-binding fragments, variants, or derivatives thereof described herein will not elicit a deleterious immune response in the animal to be treated, e.g., in a human. In certain embodiments, binding molecules, e.g., antibodies, or antigen-binding fragments thereof described herein are derived from a patient, e.g., a human patient, and are subsequently used in the same species from which they are derived, e.g., human, alleviating or minimizing the occurrence of deleterious immune responses.

De-immunization can also be used to decrease the immunogenicity of an antibody. As used herein, the term “de-immunization” includes alteration of an antibody to modify T cell epitopes; see, e.g., international applications WO98/52976 and WO00/34317. For example, VH and VL sequences from the starting antibody are analyzed and a human T cell epitope “map” from each V region showing the location of epitopes in relation to complementarity determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., FcγRIII-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In some embodiments, a humanized version of the 3G8 antibody is used. 3G8 is a well-characterized mouse IgG k1 mAb specific for FγRIII (CD16). A humanized 3G8 antibody will be as generated using standard methods. The most common method, as described for FcγRIIA mouse antibody, IV.3 (Chen et al. Ann Rheum Dis 78, 228-237 (2019)), is the grafting of the complementary determining regions (CDR) of the heavy and light chains of the mouse 3G8 antibody on to the closest human germline variable heavy and variable kappa chain genes. As the CDR grafting may result in a loss of affinity to the epitope (Safdari et al., Biotechnol Genet Eng Rev 29, 175-186 (2013)), variants with improved binding will be generated by amino acid substitutions in the CDR-grafted domain. These substitutions may follow those identified for a humanized bispecific EGFR×CD16 antibody that used 3G8 as one of the source antibodies (Asano et al., FEBS J 279, 223-233 (2012)).

Methods for conjugating antigens to antibodies are also known in the art. For example, an antibody polypeptide described herein may comprise, consist essentially of, or consist of a fusion protein. These fusion proteins are chimeric molecules which comprise, for example, an immunoglobulin FcγRIII-binding antibody and at least one heterologous tumor antigen sequence. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an antibody, or an antigen-binding fragment, variant, or analog thereof is derived from a non-immunoglobulin polypeptide of the same species, or an immunoglobulin or non-immunoglobulin polypeptide of a different species.

As discussed in more detail elsewhere herein, antibodies, or antigen-binding fragments, variants, or derivatives thereof described herein may further be recombinantly fused to a heterologous polypeptide at the N- or C-terminus or chemically conjugated (including covalent and non-covalent conjugations) to polypeptides or other compositions.

Also, a given antibody may contain many types of modifications. Antibodies may be branched, for example, as a result of ubiquitination, and they may be cyclic, with or without branching. Cyclic, branched, and branched cyclic antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination; see, e.g., Proteins—Structure and Molecular Properties, T. E. Creighton, W. H. Freeman and Company, New York 2^{nd} Ed., (1993); Posttranslational Covalent Modification of Proteins, B. C. Johnson, Ed., Academic Press, New York, pgs. 1-12 (1983); Seifter et al., Meth. Enzymol. 182 (1990), 626-646; Rattan et al., Ann. NY Acad. Sci. 663 (1992), 48-62).

Those skilled in the art will appreciate that the antibody-antigen conjugates can be fusion proteins, or can be chemical conjugates assembled using a variety of techniques. Techniques for conjugating various moieties to an antibody, or antigen-binding fragment, variant, or derivative thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2^{nd} Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation and Cytotoxic Properties of Antibody-Toxin Conjugates”, Immunol. Rev. 62 (1982), 119-158. For example, common coupling methods can be used to link antibodies to antigens via free amino, carboxylic acid, or sulfhydryl groups. Some commonly used cross-linking reagents include glutaraldehyde (links molecules to N-terminus of peptides), carbodiimide (EDC) (attaches to C-terminus of peptide); succinimide esters (e.g., MBS, SMCC) (binds free amino group and Cysteine residues); benzidine (BDB) (links to Tyrosine residues), periodate (attaches to carbohydrate groups); isothiocyanate; carbodiimide/activated ester (EDC/NHS) coupling; A reactive azide group can be site specifically introduced to the protein surface using enzymatic ligation as a posttranslational modification, which can in turn be conjugated to an alkyne-containing polymer using highly efficient click chemistry. See, e.g., Paeth et al., Methods Enzymol. 2017; 590:193-224; Leung et al., Antibodies (Basel). 2020 March; 9(1): 2. As one example, conjugates with biotin can be prepared, e.g., by reacting an FCγRIII-antibody polypeptide with an activated ester of biotin such as the biotin N-hydroxysuccinimide ester.

The antibody-antigen conjugates described herein can also be fused to other heterologous polypeptides, e.g., to increase the in vivo half-life of the polypeptides. For example, in some embodiments, PEG can be conjugated to the antibodies described herein to increase their half-life in vivo; see, e.g., Leong et al., Cytokine 16 (2001), 106-119; Adv. In Drug Deliv. Rev. 54 (2002), 531; or Weir et al., Biochem. Soc. Transactions 30 (2002), 512.

Combination with Immunotherapies: Immune Checkpoint Inhibitors

The AACs described herein can synergize with other immunomodulatory pharmaceutical agents. Thus, the present methods include administering one or more immunotherapy agents, e.g., immune checkpoint inhibitors, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4, or an antibody that binds to T-cell immunoglobulin and ITIM domains (TIGIT). In some embodiments, an anti-PD-1 antibody is used.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP 001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, Mabs. 2016 January; 8(1):10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-L1; exemplary PD-L1 protein sequences are provided at NCBI Accession No. NP 001254635.1, NP_001300958.1, and NP 054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1):80-96; Antoni et al., Am Soc Clin Oncol Educ Book. 2016; 35: e450-8; US PGPub. Nos. 20180326054; 20180251767; 20180230431; 20170334995; 20170290914; 20170101472; 20170022273; 20160303124, and include BMS-986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. See, e.g., Harjunpää and Guillerey, Clin Exp Immunol 2019 Dec. 11[Online ahead of print], DOI: 10.1111/cei.13407; 20200062859; and 20200040082.

### Pharmaceutical Compositions and Methods of Administration

The methods described herein can include the use of pharmaceutical compositions comprising an anti-FcγRIII antibody-antigen complex (AAC) as an active ingredient, and compositions comprising anti-PD1 antibodies as an active ingredient. In some embodiments, the AAC and anti-PD1 antibodies are combined in a single composition for co-administration, or are administered in separate compositions (e.g., the AAC is given first and then anti-PD1 at different intervals followed by a repetition of this regimen).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic agents, and the like, compatible with pharmaceutical administration. Supplementary active compounds can also be incorporated into the compositions, e.g., anti-PD1 can be included in the FcgRIII antibody-antigen conjugate immunization. The compositions can also include an adjuvant to increase T cell response. For example, nanoparticles that enhance T cell response can be included, e.g., as described in Stano et al., Vaccine (2012) 30:7541-6 and Swaminathan et al., Vaccine (2016) 34:110-9. See also Panagioti et al., Front. Immunol., 16 Feb. 2018; doi.org/10.3389/fimmu.2018.00276. Alternatively or in addition, an adjuvant comprising poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), and/or MPLA can also be used. See, e.g., Coffman et al., Immunity. 2010 Oct. 29; 33(4):492-503; Martins et al., EBioMedicine 3:67-78, 2016; Del Giudice, Seminars in Immunology, 2018, doi.org/10.1016/j.smim.2018.05.001; and Aurisicchio et al., Journal of Experimental & Clinical Cancer Research 2018; 37:86.

Pharmaceutical compositions are typically formulated to be compatible with its intended route of administration. Examples of routes of administration include parenteral, e.g., intravenous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the Examples below.

### Mice

All mice are on a C57Bl/6 background. Neutrophil humanized FcγR mice (Tsuboi et al, Immunity 2008) are γ^{-/-} mice lacking all endogenous mouse activating FcγRs that express human FcγRs, FcγRIIIB (CD16B) and FcγRIIA (CD32A) selectively on neutrophils (CD16B-CD32A/γ^{-/-}). OT-I mice express a transgenic T cell receptor (TCR) recognizing Ovalbumin (Ova) residues 257-264 (SIINFEKL) in the context of H2K^{b} on CD8 T cells (The Jackson Laboratory #003831). OT-I/βactin-GFP were obtained by crossing OT-I mice with β actin-GFP mice. Animals were maintained in a specific pathogen-free facility. All in vivo experiments were conducted with age and sex matched animals. The Brigham and Women's Hospital Animal Care and Use Committee approved all procedures in this study.

### Reagents

Anti-CD16B (3G8) or mouse IgGκ (non-targeting isotype control for the CD16B antibody) (Biolegend) were biochemically conjugated to FITC-Ova (#O23020, Thermofisher) as a custom order (Biolegend) to generate AAC and IgG-Ova, respectively. H-2 K^{b} Ova Tetramer (Ova257-264) was from the NIH Tetramer Core Facility, and anti-PD-1 (clone RMP1-14) and Isotype control were from BioXcell.

### B16F10-Ova Tumor Challenge and Treatment

B16F10 cells expressing soluble Ova were cultured in vitro in DMEM/high glucose supplemented with 10% fetal calf serum and maintained at sub-confluent density. Mice were anesthetized, shaved and injected in the flank subcutaneously with 1×10⁵ tumor cells in 100 μl HBSS. Tumors were measured with a caliper every 2 days once palpable in any one group (long diameter and short diameter) and tumor volume was calculated using an ellipsoid formula (½×D×d²) where “D” and “d” are the longer and shorter diameter respectively. On day 5 after tumor inoculation, mice were injected with 1×10⁶ naïve CD8⁺ T-cells isolated from the spleen and lymph nodes of OT-I mice using a negative selection kit (Miltenyi Biotec). On day 6, AAC (10 ug) or IgG-Ova (10 ug) were injected i.v. via the retro-orbital plexus, and anti-PD-1 (250 ug) was given intraperitoneally at the times indicated in the schematic.

### Tumor Harvest and Flow Cytometric Analysis

Tumors were surgically removed and processed. The tumors were gently dissociated in FACS buffer (PBS supplemented with 2% FCS and 2 mM EDTA) by shearing the tissue on a 70 μm nylon cell strainer (FisherBrand) using a 3 ml syringe plunger. Tumors were minced/digested in Collagenase type I and dissociated using gentle MACS Dissociator 1× and resuspended in FACS buffer. Cells were subjected to red blood cell lysis using ACK lysis buffer solution (Lonza Cat 10-548E) for 2 min at room temperature, washed once with PBS and resuspended in FACS buffer. Single-cell suspensions were stained with eBioscience Fixable Viability Dye and specific fluorophore-labelled antibodies to surface and intracellular markers to detect NK cells and T cell subsets and their phenotypes by flow cytometry: CD45 for immune cells, CD4 and CD8 for T cells, NK1.1 and CD49b for NK cells, MHC-I^{tet} (H-2 K^{b} Ova Tetramer, Ova₂₅₇₋₂₆₄) for Ova-specific CD8 T cells, granzyme B, perforin, IFNγ and TNFα for intracellular markers of CD8 T cell effector functions, PD-1 and TCF1 for identification of T_{SCM}, and CD103 and CD69 for identification of T_{RM}. For intracellular staining, cells were cultured with Cell Activation Cocktail with Brefeldin (Biolegend) for 5 hrs at 37 °C, then stained with surface markers followed by fixation and permeabilization with BD Perm/Wash Buffer (BD Biosciences). Cells were then stained with intracellular antibodies. Cells were washed again with permeabilization buffer and analyzed by a Symphony flow cytometer (BD Biosciences). MHCI^{tet} were from the NIH Tetramer Core Facility. All other antibodies were from Biolegend.

### Example 1. Anti-CD16B-Antigen Conjugate (AAC) but not Anti-PD-1 Treatment Increases Early T Cell and Natural Killer (NK) Cell Accumulation in an Established B16F10-Ova Melanoma

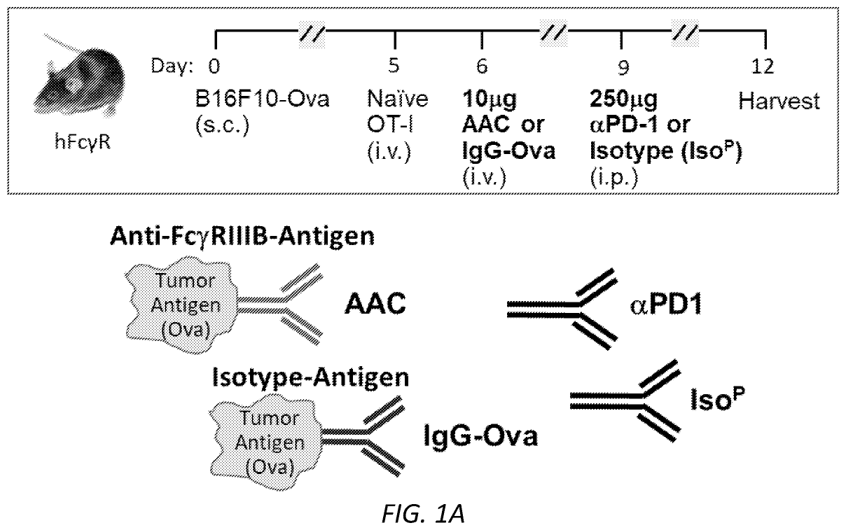

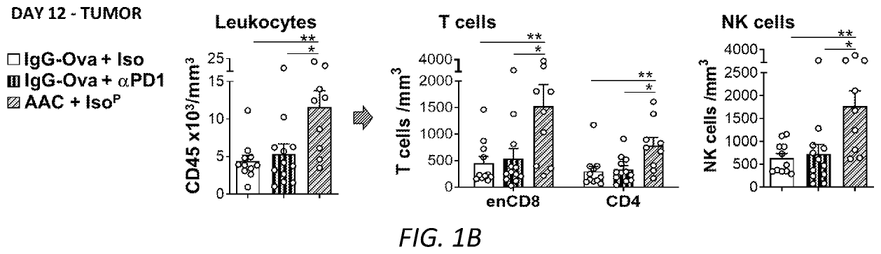

Our previous data showed that an anti-CD16B-antigen conjugate (AAC) targeting neutrophils induced antigen-specific CD8⁺ and CD4⁺ T cell proliferation, differentiation and IFNγ generation in the spleen and lymph nodes of immunologically naïve CD16B-CD32A/γ^{-/-} mice (Mysore et al, Nature Commun 2021). The antigen used in our studies is Ovalbumin (Ova), a model T cell dependent antigen. Here, we examined if AAC induces T cell infiltration in an established “hard-to-treat” solid tumor, B16F10 melanoma expressing Ova. At day 5 after subcutaneous seeding of B16F10-Ova cells in CD16B-CD32A/γ^{-/-} mice, isolated naïve GFP-Ova-specific CD8⁺ T cells were given intravenously, followed by the intravenous (i.v.) injection of AAC or IgG-Ova on day 6 and the intraperitoneal (i.p.) injection of anti-PD-1 or isotype antibody on day 9. Tumors were harvested at day 12 and subjected to flow cytometric analysis (FIG. 1a). Treatment with AAC resulted in 2-3-fold higher intratumoral infiltration of CD45⁺ leukocytes, CD4⁺ and CD8⁺ T cells, and Natural Killer (NK) cells compared with the IgG-Ova or anti-PD-1 treated groups (FIG. 1b). These data indicate that AAC but not anti-PD-1 increases early T cell and NK cell accumulation in an established B16F10-Ova melanoma thus reprogramming the immunosuppressive tumor microenvironment, a major goal of immunotherapy.

### Example 2. AAC Reduces Tumor Growth Independently and Significantly Enhances the Efficacy of Anti-PD-1

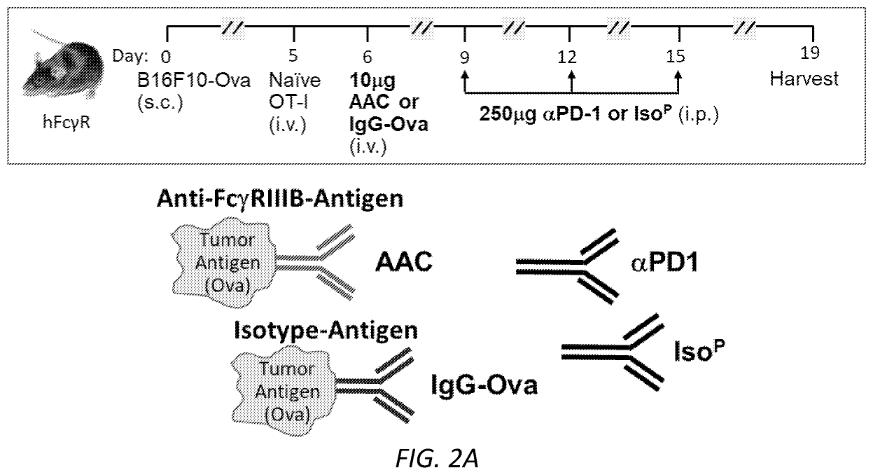

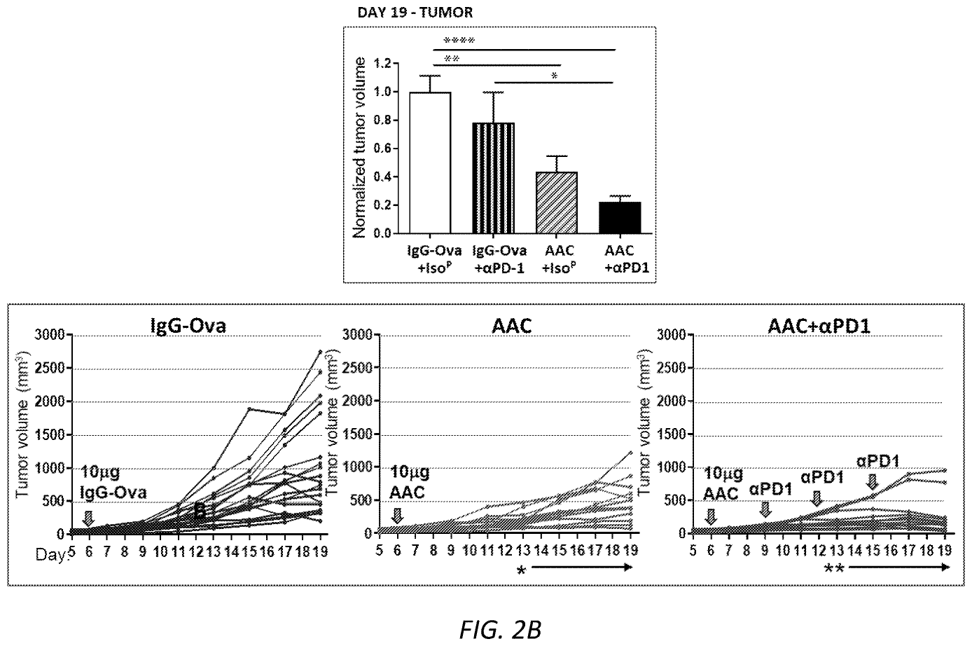

The observed AAC induced early increase in T cell and NK cells in an established B16F10-Ova melanoma (FIG. 1b) predicts that AAC would reduce tumor growth and improve the efficacy of immune checkpoint inhibitors such as anti-PD-1, which rely on pre-existing intratumoral T cells. At day 5 after B16F10-Ova seeding, CD16B-CD32A/γ^{-/-} mice were injected with naïve Ova-specific CD8⁺ T cells, followed at day 6 by AAC or IgG-Ova and anti-PD-1 or isotype antibody at day 9, 12 and 15. Melanoma was measured in individual mice with calipers over time and harvested at day 19 after euthanizing animals (FIG. 2a). The tumor volume in AAC treated mice was substantially reduced compared to the IgG-Ova treated group. Anti-PD-1 alone did not significantly affect tumor growth, while AAC in combination with anti-PD-1 resulted in a marked reduction in tumor volumes (FIG. 2b). These results demonstrate that AAC therapeutically reduces B16F10-Ova tumor growth independently and significantly enhances the efficacy of anti-PD-1.

### Example 3. AAC in Combination with Anti-PD-1 Increases Tumor Accumulation of Antigen-Specific Effector CD8⁺ T Cells, and T_{SCM} and T_{RM} Memory T Cells

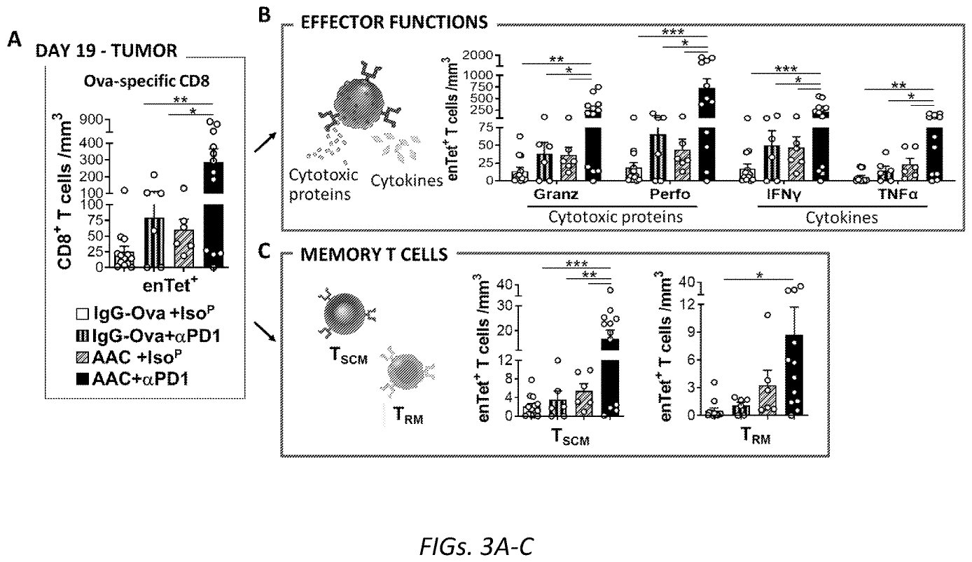

Tumors harvested at day 19 (FIG. 2a) were analyzed for tumor-infiltrating immune cell populations by flow cytometry. A larger number of Ova-specific CD8⁺ T cells (FIG. 3a) and those expressing markers of effector functions (granzyme B, perforin, IFNγ and TNFα) (FIG. 3b) were observed in mice treated with a combination of AAC plus anti-PD-1 compared to those given IgG-Ova control, AAC or anti-PD-1. The same was observed for Stem Cell Memory T cells (T_{SCM}, PD-1⁺ TCF1^{hi}) and Tissue-Resident memory T cells (T_{RM}, CD103⁺CD69⁺) (FIG. 2b), which are known to associate with anti-tumor immunity.

In addition, AAC administration in combination with anti-PD1 increased epitope spreading; in a mouse model of poorly immunogenic melanomas, spreading of epitope recognition toward wild-type melanocyte antigens has been shown to be associated with markedly improved anti-PD-1 efficacy (Lo et al., Sci Transl Med. 2021 Feb. 17; 13(581):eabd8636).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising (i) a conjugate comprising a tumor antigen and an antibody comprising an antigen-binding domain that binds to FcγRIII, and (ii) an immune checkpoint inhibitor (ICI).

2. The composition of claim 1, wherein the tumor antigen is shown in Table A.

3. The composition of claim 1, wherein the one or more ICIs are selected from an antibody that binds to PD-1, CD40, PD-L1, Tim3, Lag3, CTLA-4, or T-cell immunoglobulin and ITIM domains (TIGIT).

4. The composition of claim 3, wherein the ICI is an antibody that binds PD-1.

5. The composition of claim 1, for use in a method of treating a subject who has cancer.

6. The composition for the use of claim 5, wherein the subject has a solid tumor.

7. The composition for the use of claim 6, wherein the tumor does not have infiltrating leukocytes.

8. The composition for the use of claim 7, wherein the tumor is melanoma, pancreatic cancer, cancer, colon, glioblastoma, or ovarian cancer.

9. A method of treating a subject who has cancer, the method comprising administering to the subject an effective amount of the composition of claim 1.

10. A method of treating a subject who has cancer, the method comprising administering to the subject an effective amount of (i) a conjugate comprising an antigen and an antibody comprising an antigen-binding domain that binds to FcγRIII, and (ii) an immune checkpoint inhibitor (ICI).

11. The method of claim 10, wherein the tumor antigen is shown in Table A.

12. The method of claim 10, wherein the one or more ICIs are selected from an antibody that binds to PD-1, CD40, PD-L1, Tim3, Lag3, CTLA-4, or T-cell immunoglobulin and ITIM domains (TIGIT).

13. The method of claim 12, wherein the ICI is an antibody that binds PD-1.

14. The method of claim 10, wherein the subject has a solid tumor.

15. The method of claim 14, wherein the tumor does not have infiltrating leukocytes.

16. The method of claim 15, wherein the tumor is melanoma, pancreatic cancer, prostrate cancer, glioblastoma, or ovarian cancer.
